(19) United States
(12) Patent Application Publication
(10) Pub. No.: US 2010/0138942 A1
(43) Pub. Date: Jun. 3, 2010

(54) USE OF THE AUGMENTER OF LIVER REGENERATION PROTEIN AS AN APOPTOSIS REGULATOR

(76) Inventors: Lorenzo Polimeno, Bari (IT); Antonio Francavilla, Bari (IT)

Correspondence Address: Lonnie R Drayer, 6504 Cathedral Oaks Drive, Plant City, FL, 33565, US

(21) Appl. No.: 12/599,060

(22) PCT Filed: May 10, 2007
(86) PCT No.: PCT/IB07/01208

§ 371 Date: Jan. 12, 2010

(51) Int. Cl. A61K 49/00; C07K 14/00; C07K 16/00; A01K 67/00; A61P 25/28

(52) U.S. Cl. 800/3; 530/350; 530/387.1; 800/9

(57) ABSTRACT

The invention relates to a new use of the protein called Augmenter of Liver Regeneration (Alrp) in the regulation and control of apoptosis in mammals.

### SUMMARY OF THE INVENTION

The present invention relates to a new use of the protein called “Augmenter of Liver Regeneration” (Alrp), more specifically the invention is based on the effects of Alrp in the regulation and control of apoptosis in mammals.

### TECHNICAL BACKGROUND

It has recently been demonstrated that a wave of liver cell apoptosis accompanies cell proliferation to fine-tune the recovery of liver mass after 70% partial hepatectomy (PH). This event occurs in many biological models involving tissue growth where proliferation and apoptosis are coupled to “physiological” control of the tissue's reconstitution process. It is well known that during liver regeneration two distinct phases of apoptosis can be identified: an early phase (6 hrs after PH, early G₁) when anti-apoptotic genes (Bcl-2, Bcl-x) are induced and a late phase (20-24 hours after PH) when pro-apoptotic genes (Bax) are up-regulated. Despite these data, the regulatory agents of apoptosis, among the myriad of factors that normally increase in serum during the different phases of liver regeneration after PH, have not been identified. These have been divided into two categories: Initiators and Augmenters. The Initiators are those factors, such as ions, hormones and growth molecules, able to sustain the entire cell cycle; the Augmenters are those molecules that support the passage from the G₁ to the S phase of the cell cycle and are able to “augment” cell proliferation only in vivo and when it has already been triggered, as in Eck's fistula in dogs and in 40% PH in rats. The best known and most studied Augmenters are insulin-like growth factors-II (IGF-II), insulin (Ins) and Augmenter of Liver Regeneration (ALR).

The Augmenter of Liver Regeneration protein (Alrp) is a polypeptide expressed by a nucleotide sequence isolated from rats and humans and stimulating of DNA synthesis in hepatocytes. Said protein was first disclosed in EP668291. In this document it is reported the effect of Alrp on hepatocyte proliferation but no explanation of the possible mechanism of action is given.

To date, none of the Augmenters has been tested as a regulator of apoptosis in an experimental model of physiologically liver regeneration.

The present inventors investigated the role of Alrp on apoptosis in an experimental model of liver regeneration in rats and found out, as an unexpected result, that Alrp is deeply involved in the control of apoptosis.

This finding is extremely valuable in view of the several possible pharmacological, medical and diagnostic applications wherein Alrp, as well as the sequences encoding therefore, can be advantageously exploited.

### DESCRIPTION OF THE INVENTION

According to one of its aspects, the present invention relates to the use of the Augmenter of Liver Regeneration protein, herein after only referred to as Alrp, as a regulator of apoptosis.

According to another of its aspects, the invention relates to the use of Alrp for the preparation of a medicament for the regulation of apoptosis in mammals.

According to another of its aspects, the invention relates to the use of Alrp for the preparation of a medicament for the treatment and/or prevention of a disease wherein a regulation of apoptosis is beneficial.

According to another of its aspects, the invention relates to the use of Alrp for the preparation of a medicament for the treatment and/or prevention of a disease provoked by troubles in the apoptosis control.

According to another of its aspects, the invention relates to the use of Alrp for the preparation of a medicament for the treatment and/or prevention of a disease wherein an up-regulation of apoptosis is beneficial.

According to another of its aspects, the invention relates to a method for the treatment of apoptosis-related diseases which comprises administering, in a subject in need thereof, an effective amount of Alrp.

The term “Alrp” designates, according to the present invention, any Augmenter of Liver Regeneration protein, i.e. a Alrp deriving from any mammal.

According to a preferred aspect, Alrp is recombinant Alrp, more preferably human recombinant Alrp.

Alrp proteins, as well as the nucleotide sequences encoding thereof, are disclosed in EP668291.

The terms “regulator” or “regulation”, according to the present invention, are use to intend the control of an altered apoptosis condition.

The term “apoptosis” designates, according to the present invention, the physiologically programmed death of a cell.

The term “disease” designates, according to the present invention, any impairment, disorder, syndrome affecting a mammal.

The term “disease provoked by troubles in the apoptosis control” designates, according to the present invention, any disease which is at least partially resulting from a improper or incorrect control of apoptosis in a mammal.

Typical diseases provoked by troubles in the apoptosis control are for example degenerative diseases, such as neuro- and/or muscular-degenerative diseases, as well as diseases affecting testes.

For the use according to the present invention, Alrp must be administered in a mammal by any convenient administration route.

Preferred administration routes are for example oral or parenteral routes such as intravenous, intramuscular, subcutaneous, intraperitoneal, etc.

The administration of Alrp may be repeated as necessary, until the desired effect is achieved.

For the administration Alrp is preferably formulated in a pharmaceutical or veterinary composition, according to the methods well known to the skilled in the art.

The use of a pharmaceutical or veterinary composition for the treatments as above defined, constitute another subject-matter of the present invention.

The doses of Alrp to be administered may vary with the conditions, age and weight of the subject to be treated, administration route or dosage form, and can be conveniently be chosen by the expert physician. Anyway, as the protein is very active, dosage ranges will normally vary between picograms and nanograms per day.

According to another of its aspects, the present invention relates to the use of Alrp in the diagnostic field, for instance as a marker for detecting the presence of a proliferative disease in a mammal.

According to another of its aspects, the present invention relates to the use of an Alrp encoding sequence for the preparation of immortal cell lines, which are useful for in vitro experimental test.

According to another of its aspects, the present invention relates to the use of Alrp in the preparation of cell lines.

To this purpose, a desired cell can be transformed with the convenient ALR sequence which will express the corresponding Alrp, according to the methods known in the art.

Alternatively, Arlp itself may be provided to a desired cell, or cell line, in order to make it proliferate as necessary.

ALR gene sequences can also be used to transform non-human mammals, according to the known methods, which can be very valuable in the study of diseases involving apoptosis regulation.

So, according to another of its aspects, the present invention relates to the use of a transgenic non-human mammal transformed with an ALR gene sequence, for experimental studies on apoptosis, for instance, as a model for experimental tests on apoptosis-linked diseases.

The transgenic non-human mammal is for example a rat or a mouse.

The effect of Alrp on apoptosis was demonstrated by the present inventors by means of an experimental animal model that has allowed to assess, separately, the “proliferative” and “apoptotic” properties of growth factors (GFs) involved in a tissue regenerative process.

The data obtained from the testing, carried out as set forth in the experimental section herein below, show that, among the tested GFs, only Alrp, among the Augmenter family, inhibits apoptosis during the process tissue regeneration. More particularly, it has been shown that the effect of Alrp on apoptosis involves the Stat3 activation (dimerization and phosphorylation), the induction of anti-apoptotic genes and, more relevant, the down-regulation of pro-apoptotic genes. A similar effect has not been demonstrated for Ins and IGF-II, the others two members of the Augmenter family.

According to another of its aspects, the present invention relates to the use of antibodies anti-Alrp for the treatment of a disease wherein a down-regulation of apoptosis is beneficial.

Typical diseases wherein a down-regulation of apoptosis is beneficial are for example cancer and other proliferative diseases, such as for instance psoriasis, scleroderma, etc.

According to another of its aspects, the present invention relates to the use of antibodies anti-Alrp in the diagnostic field, for detecting the presence of Alrp.

### Experimental Section

The present application based was supported by two different experimental approaches: The first was (i) to set up a “physiological” animal model necessary to visualize the biological effect of Alrp on hepatocyte apoptosis phenomenon, the second (ii) to demonstrate the biochemical pathway involved in the biological process controlled by Alrp.

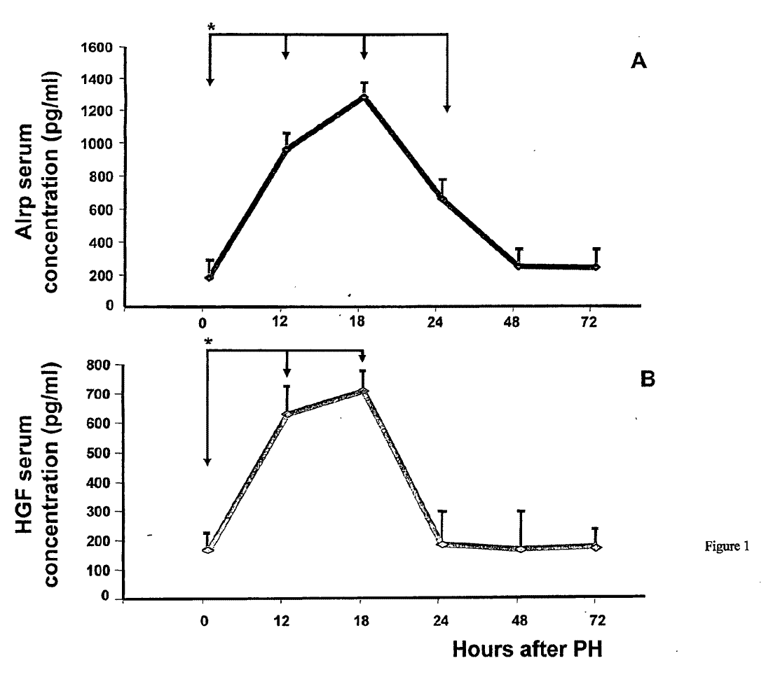

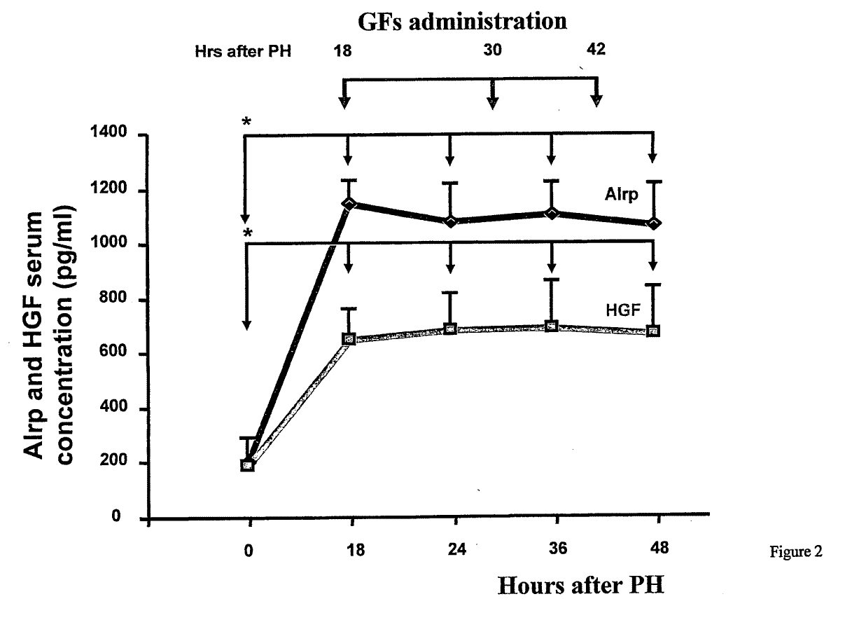

(i) To organize the model, we first determined serum profiles of HGF and Alrp in 70% partial hepatectomy (PH) rats (Tomiya T et al., 1998, Am. J. Pathol. 153, 955-61; Gandhi C R et al, 1999, Hepatology 29(5), 1435-4), recording a peak for both GFs at the 18^{th} hr after the surgery, followed by a steady rapid decline that continued until the 72^{nd} hr (FIG. 1). On this basis, in a different group of 70% PH rats, we administered rHGF or rAlrp six hourly before their “physiological” decline maintaining serum levels as high as those found at the time point of their maximal expression (18^{th} hour) throughout the period of observation (48 hrs after PH) (FIG. 2).

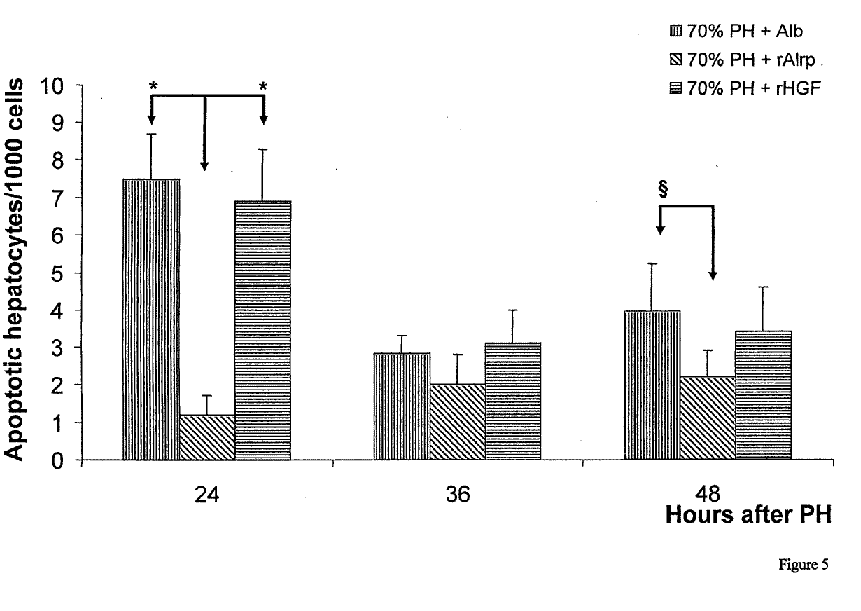

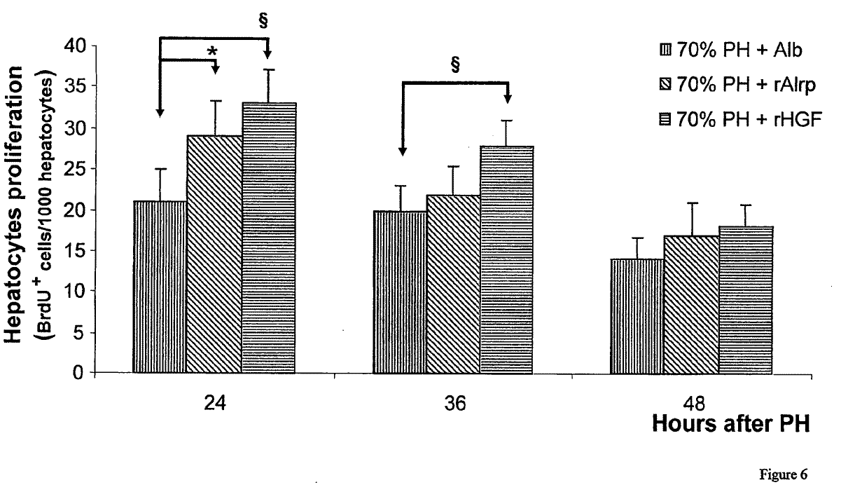

The administration of rHGF only moderately increased hepatocyte proliferation (FIG. 6) with no effect on hepatocyte apoptosis (FIG. 5), while rAlrp injection dramatically reduced programmed cell death (FIG. 5) with only a mild effect on cell proliferation (FIG. 6). Further support for an anti-apoptotic effect of rAlrp comes from the study of the apoptosis-related genes with an up-regulation of the anti-apoptosis gene (Bcl-2) and a marked down-regulation of the pro-apoptosis genes (Bax and Caspase 3), effect that is known to be mediated by Stat-3.

(ii) Stats proteins are a family of cytoplasmic transcription factors present in many cell types and activated by a variety of cytokines such as IL-6, IL-2 and IL-10, granulocyte CSF, EGF, PDGF, IFN-γ, and leptin (Takeda k et al 1998, Schindler C and Darnell J E Jr., 1995, Akira S. 1997). Stats activation, induces transcriptional regulation of many genes involved in apoptosis, cell growth and related survival (Darnell J. E, Jr 1997; Stark G R et al, 1998; Levy D E et al., J. Clin. Invest., May; 109(9):1143-8.2002; HiranoT, et al. Oncogene, 2000 May 15; 19(21):2548-56). Among Stats proteins family, Stat-1 and Stat-3 are the most studied. These two proteins, even if share a similarity of 40% (Shen Y et al., PNAS, 2001 13, 98) and are often activated by the same ligands, exert different gene regulation. In particular Stat-1 controls pro-apototic gene expression, such as caspase-1, -2, -3, Stat-3 modulates cell proliferation and apoptosis by up-regulating genes encoding apoptosis inhibitors, such as Bcl-2, Surviving, Bcl-XL (Bromberg J F et al, 1999; Haga S et al, 2003, J Clin Invest. 2003 October; 112(7):989-98; Taub R, J Clin Invest. 2003 October; 112(7):978-80; Dolled-Filhart, Clin Cancer Res. 2003 February; 9(2):594-600, Darnell et al, 1994; Bromberg et al, 1998; Sailor et al, 1997; Garcia et al, 2001; Song H. Proc Natl Acad Sci USA. 2005 Mar. 29; 102(13):4700-5).

Recently it has been proposed also that Stat-3 activation may be involved in oncogenesis by stimulating cell proliferation and conferring resistance to apoptosis (Catlett-Falcone et al, 1999a, 1999b; Alas et al, 2003; Wei et al, 2003). In fact, blockade of activated Stat-3 inhibits cancer cell growth (Alas et al, 2003; Aoki et al, 2003; Epling-Burnette 2001), and interruption of Stat-3 activity leads to apoptosis (Darnell et al, 2005; Bromberg et al, 1999). Stats proteins are normally activated when ligands bind their specific cell-surface receptors and activate tyrosine kinases (Levy et al, 2002). Stats become activated by tyrosine phosphorylation, dimerize through SH2-phosphotyrosine interaction and accumulate in the nucleus where activate gene transcription. Nevertheless, recently many studies refer that Stat3 dimerization can take place without previous phosphorylation; many laboratories provided evidence that STATs proteins exist as dimers. Specifically Kretzschmar demonstrated the existence of non-phosphorylated STAT3 dimers. Shen and collegues demonstrated that a constitutively activated form of Stat-3, (Stat-3 C), product of Stat-3 dimerization (Lange et al, 2000; Shen Y et al., PNAS, 2001 13, 98), can cause cellular transformation of fibroblasts, that become more resistant to pro-apoptotic stimuli than non-transformed cell. Novak et al since 1998 reported the existence of Stat-3 non-posphorylated dimers in cells that allow for cysteine-cysteine cross-link, due to an enzymatic control by a sulfydryl oxidase enzyme.

All these data support the idea that Stat-3 phosphorylation and dimerization, events necessary for its activation, are two biochemical processes that can take place in the cytoplasm separately and not necessarily in this sequence. Stat-3 can be present in the cytoplasm as non-phosphorylated homodimer, specifically oxidated in SH— groups by a devoted enzyme with sulfydryl oxidase activity. The only problem in this respect is that the existence of a specific sulfydryl oxidase enzyme responsible of Stat-3 dimerization has not been identified until now.

Recently we reported that rat and human Alrp are flavin-linked sulfydryl oxidase that catalyze the formation of disulfide bonds in reduced protein substrate (Lisowsky T., Dig. Liv. Dis., 2001, 33:173-80). Indeed the present inventors already reported the Alrp-sulfydryl oxidase enzymatic activity related with a conserved CXXC motif in the carboxy-terminal domain present in the homologous proteins from yeast to humans (Polimeno L. Ital. J. of Gastroenter. and Hepat., 1999; Lisowsky T., Dig. Liv. Dis., 2001, 33:173-80). Nevertheless the physiological substrate for Alrp enzymatic activity is still undiscovered until now.

In the present invention the authors demonstrated that
1) Stat-3 is the physiological substrate for Alrp sulfydryl oxidase enzymatic activity, and, more important for the invention
2) Stat-3 dimerizes when incubated in presence of Alrp.

Taken together these results confirm the data presents in literature reporting the existence of non-phosphorylated Stat-3 dimers and indicate in Alrp the factor with sulfydryl oxidase activity able to control Stat-3 —SH oxidization and dimerization, cellular event that generates a molecule (Stat3 homodimer) suitable to be phosphorylated and in turn able to up-regulate apoptosis inhibitors encoding genes.

In conclusion the inventors demonstrate, by all the data on the effect of rAlrp on hepatocyte apoptosis and on Stat-3 dimerization, that Alrp controls Stat3 dimerization and activation and in turn hepatocyte apoptosis, representing the physiological component of apoptosis in the process of tissue regeneration following PH.

### Materials and Methods

### Reagents

Recombinant Alrp (Alrp) expressed in transfected COS-1 cells was produced by the Laboratory of Biochemistry and Molecular Biology, University of Georgia, Athens, Ga., USA. Full length recombinant-Stat3 was purchased from SignalChem (Richmond, Canada). Primary specific antibody for Stat3 was purchased by Upstate (NY, USA). Albumin, r-HGF and r-IGF-II were purchased from Sigma-Aldrich, Milan, Italy. Chemical reagents were purchased, if not specifically reported, by Sigma-Aldrich (Milan, Italy).

### Animals

For these experiments 225 F344 male rats, weighing 180-200 g, purchased from Charles River (Milan, Italy), were used. Animals were housed in a room with controlled light (7 a.m.-7 p.m.) and temperature (21±1° C.). Animals could eat and drink ad libitum and were cared for according to the standard procedures indicated in the “Guide for the Care and Use of Laboratory Animals” published by the National Institutes of Health (NIH 80-23). Partial hepatectomy (70%) was performed according to Higgins (Higgins, G M and Anderson, R M 1931, Arch. Pathol. 12, 186-202), under deep anaesthesia with ether. The animals were sacrificed by exsanguination and liver and blood samples taken as stated by the protocols.

### Protocols, Methods and Results

### Effect of Augmenters on Stat-3 Phosphorylation

Twenty male rats were used. All animals underwent 70% PH (Higgins G M and Anderson R M, 1931). Five were sacrificed soon after surgery (time 0 group), fifteen (divided in three groups of five animal each) were treated with a single injection of Alrp (500 ng/each animal, i.p.) or recombinant IGF-II (rIGF-II, 1000 ng/each animal, i.m.) or albumin (500 ng/each animal, i.p.) 18 hours after surgery and sacrificed 6 hrs later (24 hrs after PH). At the time of sacrifice, liver samples were taken to evaluate Stat-3 phosphorylation by immunohistochemistry (Chan K S et al, 2004).

An intense cytoplasmic and nuclear Stat-3 positivity was evident in hepatocytes of Alrp-treated rats; on the contrary, a weak positivity was detected in hepatocytes from albumin- or IGF-II-treated rats. In this experiment, among Augmenters, only IGF-II was used, since Insulin serum levels in PH rats is already elevated at the time of our observation (24 hrs after PH) without any evident effect on Stat-3 phosphorylation (control animals).

### Effect of Alrp on Stat-3 Dimerization

### Methods

### Spectroscopy of Recombinant Alrp.

With the goal to evaluate the presence of a FAD moiety bound to rAlrp on which depends its sulfidryl oxidase activity, 138 μg of rAlrp, corresponding to 9 μM final concentration, were dissolved in 1 ml of 100 mM KH₂PO₄, pH 7.5 and absorbance from 300 to 600 nm was measured with a Beckman DU 7400 spectrophotometer.

### Enzyme Assay for Sulfydryl Oxidase.

To determine Alrp sulfydryl oxidase activity, Ellman's reagent method (Lisowsky T., Dig. Liv. Dis., 2001, 33:173-80) was followed. Briefly, prior to the enzyme assay, DTT was removed from the commercially available Stat3 solution by centrifugal filter devices (Millipore Microcon YM-50). Concentrated Stat3 solution, corresponding to 0.9 nmol reduced thiol groups, was then diluted in 1 ml measurement buffer (2 M urea, 1 mM EDTA, 100 mM potassium phosphate, pH 7.5) and incubated at 37° C. together with or without 0.67 μmol rAlrp. Thiol content at different time intervals was determined by adding 10 μM (final concentration) Ellman's reagent and using an extinction coefficient of 14.15 mM⁻¹ cm⁻¹ at 412 nm.

### Stat3 Homodimer Evaluation

To identify Stat3 homodimers consequent to S—S bridges formation due to rAlrp sulfydryl oxidase activity, western blot analysis was performed after 5′ minutes incubation of DTT-removed Stat3 in presence or not of rAlrp. Briefly, DTT-removed Stat3 (8 μg), obtained by centrifugal filter devices (Millipore microcon YM-50), was incubated for 5′ at 37° C. in 1 mM EDTA, 100 mM potassium phosphate, pH 7.5 in presence or not of 200 ng of rAlrp (final volume 30 μl). After incubation the reaction mixtures were loaded on 7.5% sodium dodecyl sulfate-polyacrylamide gels (Biorad, Calif., USA). In two separate electrophoretic lines DTT-removed Stat3 (8 μg) and proteins MW markers (Biorad, Calif., USA) were contemporary loaded. At the end of electrophoresis run, the proteins were transferred on nitrocellulose membrane that was then probed with primary antibody specific for Stat3 (rabbit Upstate, N.Y., USA). The primary antibodies were detected using alkaline-phosphatase-conjugated secondary antibodies (Biorad, Calif., USA) and chemiluminescence (Amersham, England).

### Results

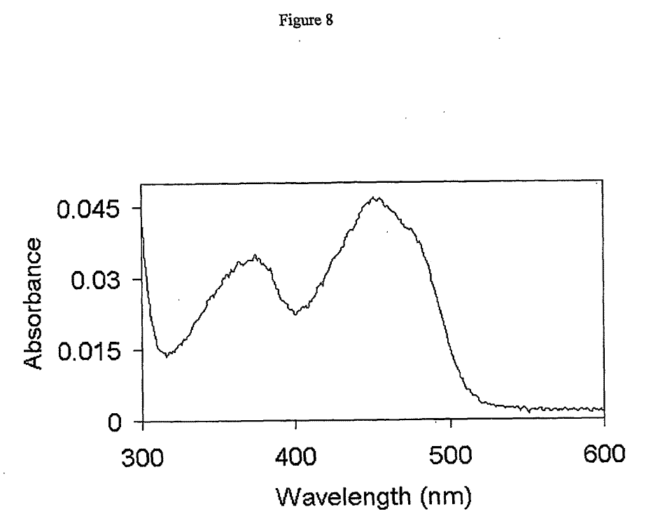

Spectroscopy of rAlrp is shown in FIG. 8. The spectrum presents two maxima at 360 nm and 450 nm respectively peculiar for protein-bond flavin (Lisowsky T., et al, DLD, 2001, 33:173-80). Using an assumed extinction coefficient of 10 mM⁻¹ cm⁻¹ at 460 nm we found 0.5 flavin molecule per monomer unit of protein where as in human cells the protein subunit/FAD stoichiometry is 1:1. FAD content of recombinant Alrp depends on the protein expression and purification procedures. These results confirm the presence of FAD linked to Alrp.

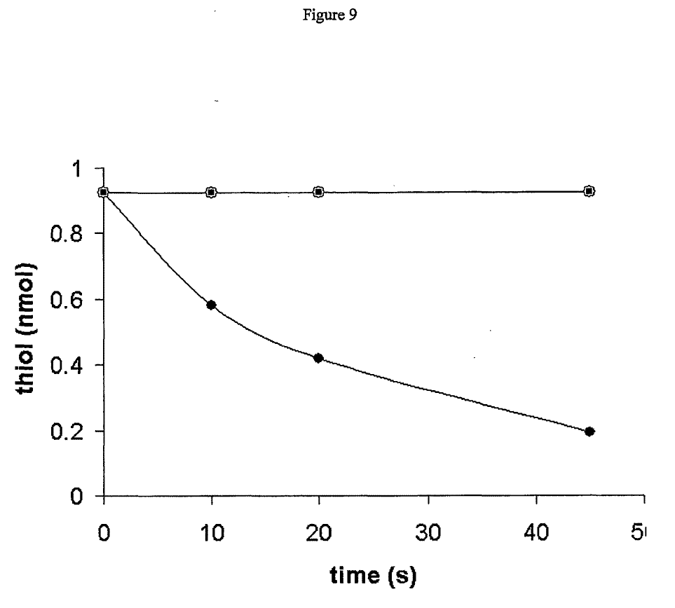

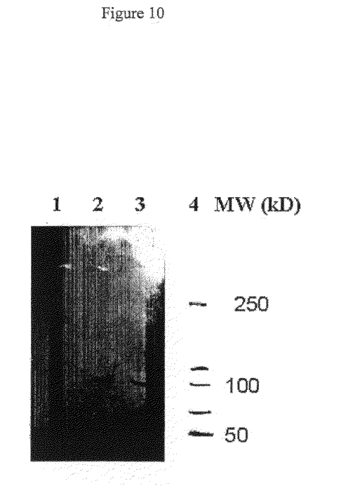

FIG. 9 reports Stat3 thiol groups disappearance when incubated in presence of rAlrp. The obtained data demonstrate that Stat3 is an excellent substrate for the knew Alrp sulfydryl oxidase enzymatic activity. One-half oxidation in fact is complete in 17 sec with 20% of thiol groups remaining after 45 sec of incubation. From the tangent to the progress curve a initial rate of 0.037 nmol thiols s⁻¹ can be estimated corresponding to 97 S—S bridge formation per active protein per sec. No thiols oxidation neither without rAlrp nor with purified FAD alone was detectable. FIG. 10 reports the homodimer formation of Stat3 when incubated in presence of rAlrp, line 1. Line 2 reports Stat3 profile when incubated in absence of rAlrp. Line 3 shows the electhophoretic profile of Stat3 as sold by the company (control) and line 4 shows the MW profile. Only in line 1 it is possible to identify an immunological recognized protein by the anti-Stat3 antibody with a MW approximately double of Stat3 protein, effect of Stat3 homodimer formation under Alrp sulfydryl oxidase enzymatic activity

Experimental Model Used to Study the Influence of GFS (rAlrp and rHGF) on Apoptosis During Liver Regeneration

### A) Alrp and HGF Serum Levels in Untreated 70% PH Rats

Thirty male rats were used. The animals, divided into six groups of five animals each, underwent 70% PH and were sacrificed at 0, 12, 18, 24, 48 and 72 hours after surgery. At the time of sacrifice, under deep ether anaesthesia, the animals were laparotomized and blood samples were taken to determine Alrp and HGF serum levels.

A significant (* p<0.001) increase in serum Alrp concentration was registered at 12 hours after surgery (5 times higher than the pre-surgery value), reaching the maximal expression (7 times higher than the pre-surgery value) 18 hours after surgery, then starting to decline at 24 hours and reaching the pre-surgery level at 48 and 72 hours (FIG. 1A).

A similar pattern was detected for the HGF serum profile. A significant increase (* p<0.001) was evident starting 6 hrs after surgery (2 times the pre-operative value), the maximal expression being reached at 18 hrs (4 times the pre-operative value). A″ constant progressive decline was then detected, the baseline value being reached at 48 and 72 hrs after surgery (FIG. 1B).

B) Alrp and HGF Serum Levels after their Administration in 70% Partially Hepatectomized Rats

On the basis of the above data we decided to inject 70% PH animals with rAlrp or recombinant HGF (rHGF) starting from 18 hrs after surgery with the goal of maintaining serum levels of the growth factors as high as at the maximal expression documented by our present results and reported in the literature. rAlrp and rHGF were used at a dosage reported in previous experiments and able to produce their biological activities. Forty rats underwent 70% PH; ten rats were divided in two groups of five animals each: five were sacrificed soon after surgery and used as time point “0 hrs” and five were sacrificed at 18 hours after surgery and used as time point “18 hrs”; the other thirty were divided in two groups of fifteen animals each. One group was injected with a multiple dose of Alrp (500 ng/rat/each injection) at 18, 30 and 42 hours after surgery and sacrificed, 5 animals each time, at 24, 36 and 48 hours after PH. The other group was treated, following the same protocol, with rHGF at a dose of 1000 ng/rat/each injection. Blood samples were taken from all animals to determine Alrp and HGF serum levels. The results obtained (FIG. 2) show that administration of the two growth factors to hepatectomized rats, starting from 18 hours after PH, was able to maintain, at least for the period of our observation (up to 48 hours after PH), serum levels as high as those observed at their maximal expression (* p<0.001 vs 0 time value).

### Hepatocyte Proliferation and Apoptosis in Untreated and GFs-Treated 70% PH Rats

### 1) Determinations in Untreated 70% Partially Hepatectomized Rats

For these experiments thirty-five male rats were used. All animals underwent 70% PH and were divided into seven groups of five rats each, sacrificed 0, 6, 12, 18, 24, 36 and 48 hours after surgery. At the time of sacrifice the rats, under deep ether anaesthesia, underwent laparotomy and the liver was excised and used to measure:
hepatocytes apoptosis by TUNEL analysis,
hepatocytes proliferation by BrdU incorporation.

### Hepatocyte Apoptosis

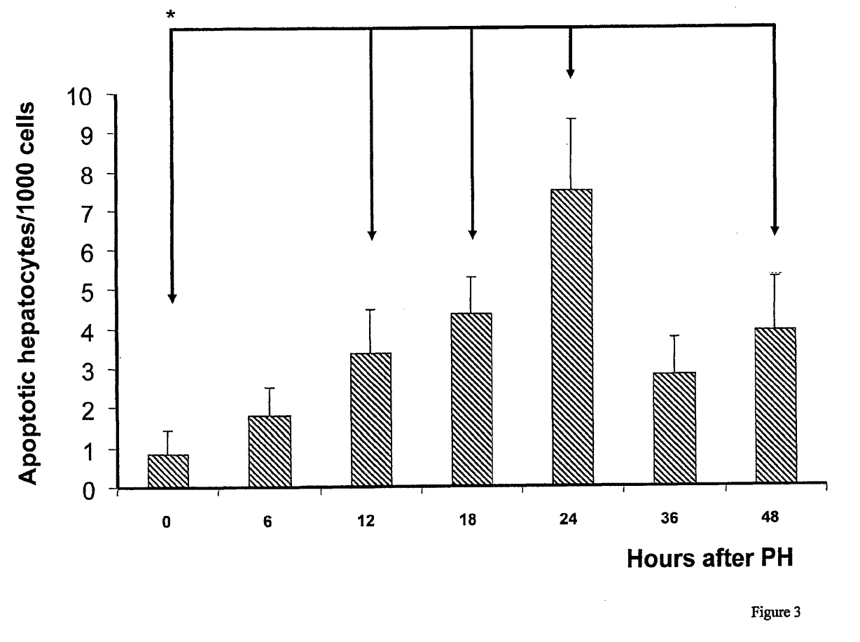

An increase in apoptotis was recorded starting from 6 hours after PH (1.8±0.7 apoptotic bodies/1000 hepatocytes) and reaching its peak at 24 hours (7.5±1.8 apoptotic bodies/1000 hepatocytes). A decrease was recorded at 36 and 48 hours (FIG. 3; *p<0.001 vs time zero).

### Hepatocyte Proliferation

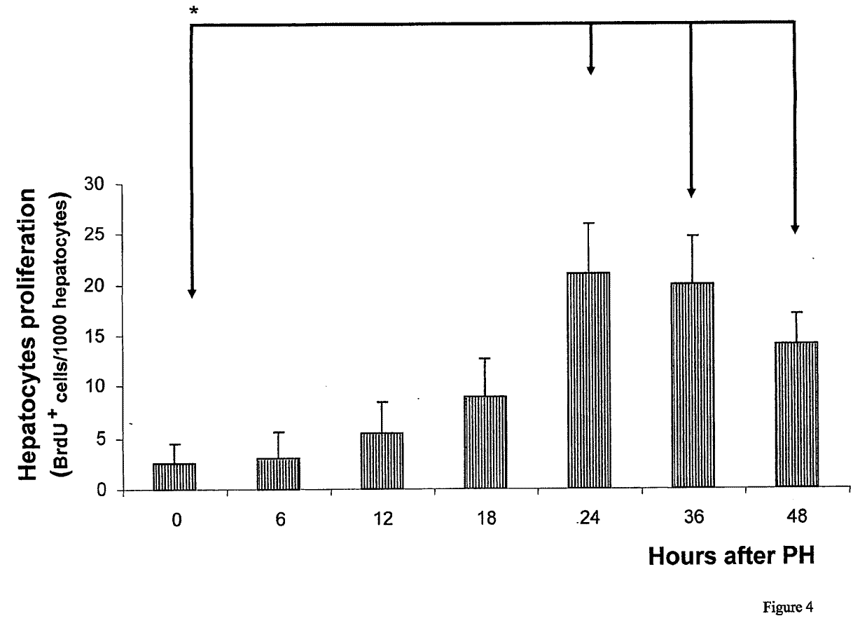

A typical pattern of hepatocyte proliferation after PH was observed. As compared to zero time point (Labelling Index=BrdU⁺ hepatocytes/1000 hepatocytes; L.I.=2.5±2) increased 12 hours after PH (L.I.=5.5±3), reached its maximal expression at 24 hours (L.I.=21±5; p<0.001) and then steadily decreased 36 hours (L.I.=20±4.7; p<0.001) and 48 hours (L.I.=14±3; p<0.001) after surgery (FIG. 4).

2) Determinations in 70% Partially Hepatectomized Rats and Alrp- or rHGF-Injected

For these experiments forty five rats were used, which underwent 70% PH and were then divided into three groups with fifteen animals each: one group was injected with Alrp (500 ng/each time/each animal, i.p.), one group with rHGF (1000 ng/each time/each animal, i.v.) and one group with albumin (500 ng/each time/each animal, i.p.). Injections were done 18, 30 and 42 hours after surgery. Five animals from each group were sacrificed at 24, 36 and 48 hours.

At the time of sacrifice, the rats, under deep ether anaesthesia, underwent laparotomy and the liver was excised and used to analyze:
hepatocytes apoptosis by TUNEL analysis,
hepatocytes proliferation by BrdU incorporation.

### Hepatocyte Apoptosis

Results are reported in FIG. 5. Albumin-treated animals (controls) showed an apoptotic profile consistent with that registered in untreated PH rats. A similar pattern was also observed in PH animals treated with rHGF. The treatment with rAlrp, on the contrary, induced a significant decrease (*p<0.001 vs controls and rHGF-treated rats) of apoptotis at 24 hours (1.1±0.5 apoptotic bodies/1000 hepatocytes) and at 48 hours (2.2±0.7 apoptotic bodies/1000 hepatocytes; ^{§}p<0.01 vs controls rats). This decrease was also evident, but not statistically significant, at 36 hours (2±0.8 apoptotic bodies/1000 hepatocytes).

### BrdU Incorporation

Hepatocyte proliferation in albumin-treated animals shows the typical profile recorded in untreated 70% PH rats (24 hrs-L.I.=22±3.5, 36 hrs-L.I.=21±2.4 and 48 hrs-L.I.=13±2.5) (FIG. 6). As compared to controls at the same time point after surgery, the administration of rAlrp and rHGF induced an increase in hepatocyte proliferation (Brdtt hepatocytes) at 24 hours (rAlrp-L.I.=29±4.2; rHGF-L.I.=33±4; p<0.01; §=p<0.001), 36 hours (rAlrp-L.I.=22±3.5; rHGF=28±3; ^{§}p<0.001) and at 48 hours (rAlrp-L.I.=17±3.9; rHGF-L.I.=18±2.7, n.s.). (FIG. 6)

Effect of rAlrp Administration on Genes of the Bcl-2 Family in 70% PH Rats

Twenty-five male rats were used for this experiment. All animals underwent 70% PH and were then divided into three groups. One group of five rats was sacrificed soon after surgery (time “0” group), a second group of ten rats was injected with multiple dose of rAlrp (500 ng/rat/each injection) at 18 and 42 hours after surgery and sacrificed, 5 animals at each time, six hrs later (at 24 and 48 hours respectively), a third group of ten rats was injected with albumin (500 ng/rat/each injection, i.p.) following the same protocol. At the time of sacrifice the rats, under deep ether anaesthesia, underwent laparotomy and the liver was excised and used to determine mRNA expression for apoptotic genes (Bcl-2, Bcl-XL, Bcl-XS, Bax, Casp-3) by Multi-PCR.

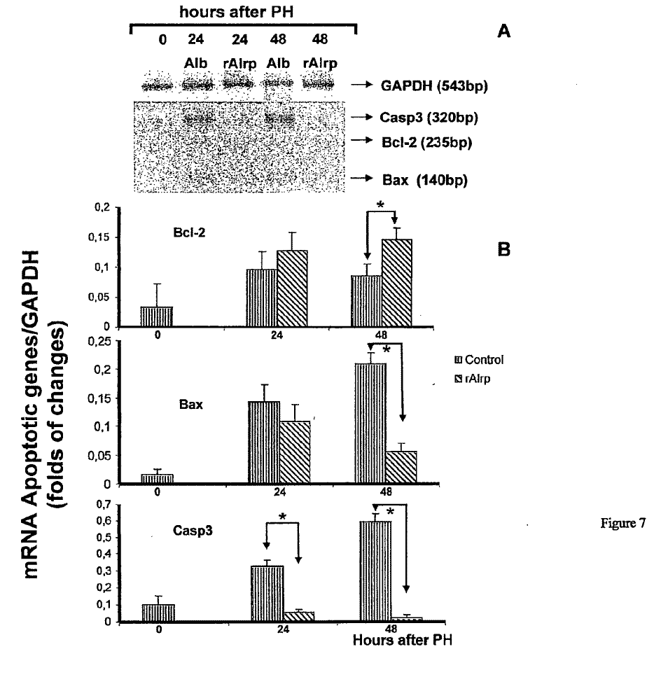

A decrease of pro-apoptotic genes (Bax and Casp-3) mRNA expression was registered in rAlrp-treated rats in comparison to albumin-treated rats (FIG. 7A). The densitometric analysis of Bax mRNA expression revealed a strong reduction in the growth factor-injected animals both at 24 and 48 hrs time points, a reduction that became statistically significant (*p<0.001 vs albumin-treated rats) at 48 hrs time point after surgery (FIG. 7B). A similar and, in some ways, stronger picture emerges evaluating the densitometric analysis of Casp-3 mRNA expression. In this case also a dramatic, statistically significant (*p<0.001 vs albumin-treated rats), decrease of the gene expression was registered both at 24 and 48 hrs after surgery. Taken together the above results (Bax and Casp-3 mRNA expression) clearly indicate a dramatic down-regulation of pro-apoptotic mRNA gene expression in rAlrp-treated rats after 70% PH.

Exactly opposite is the pattern of mRNA expression of Bcl-2, an anti-apoptotic gene. In fact an up-regulation of Bcl-2 expression was recorded both at 24 and 48 hours after PH, in rAlrp-treated rats compared to albumin-treated rats (FIG. 7A). The densitometric analysis of the signal of the amplified gene revealed a statistically significant increase (*p<0.001) between the two animals group at 48 hrs time point (FIG. 7B).

Considering these results, it is clear that the response of post-PH regenerating liver to rAlrp injection is an up-regulation of an anti-apoptotic and a down-regulation of pro-apoptotic genes.

Total RNA Extraction, RNA Preparation and First-Strand cDNA Synthesis

Total RNA was extracted from liver tissue using the Rneasy Mini Kit (Qiagen GmbH, Germany) according to the manufacturer's instructions. Final mRNA concentrations were estimated by ultraviolet (UV) absorbance at 260 nm. Aliquots of total RNA (1 μg) were reverse transcribed using random hexamers and the TaqMan Reverse Transcription Reagents (Applied Biosystems, Monza, Italy) with 3.125 U/μl of MultiScribe Reverse Transcriptase in a final volume of 50 μl.

Multi-PCR for Apoptotic Genes mRNA Determination

A Multiplex RT-PCR APO2B (Eppendorf, Milan Italy) commercial kit was used to perform multi-PCR. An optimal amount of mixed primers, specific for the ORF (Open Reading Frame) region of the studied apoptotic genes (Casp-3, Bcl-2, Bax, Bel-XL, Bel-XS) and GAPDH (Glucose-6-phosphate dehydrogenase), used as housekeeping gene, are present in the kit.

Briefly, cDNA (1 μl) of each sample was amplified in a final volume of 50 μl of the following reagent mixture: 10×rAPO2B MPCR Buffer, 10×rAPO2B Primers, Taq DNA polymerase (Perkin-Elmer, Elmer Cetus, Calif. USA) 5 U/μl, dNTPs (1 mM each). The simultaneous amplification of these genes was obtained using the “Hot start” PCR method with the following reaction profile: 94° C.×3′, 2 cycles (96° C.×1′, 68° C.×4′), 33 cycles (94° C.×1′, 68° C.×2.5°), 70° C.×10′, gradual ramp from 70° C. to 20° C. in 30′, according to the manufacturer's instructions. Aliquots (10 μl) of each sample obtained by PCR were then electrophoresed on agarose gel (4%) in the presence of ethidium bromide and photographed by Polaroid using UV trans-illuminator (SIGMA, St. Louis USA). The densitometric analysis of the signal of each amplified gene was evaluated by the Optilab System 2.0 (Graftek, Pavia, Italy). The data were reported as ratio between the densitometric value of each apoptotic gene and that of GAPDH.

### Serum GFs Determination

A standard ELISA procedure was used to evaluate serum levels of Alrp mainly following a procedure previously described in literature (Gandhi, C. R. et al., 1999, Hepatology 29(5), 1435-4); specific rabbit polyclonal antibodies anti-Alrp were used.

For HGF serum evaluation a commercial kit was used following the procedure described by the company (B-Bridge International, Inc. Mountain View, Calif. 94043 USA).

### Apoptosis Evaluation: TUNEL

Liver sections of 3 micrometers in thickness were used for detection of DNA fragments of apoptotic cells by the Terminal deoxynucleotidyl-transferase-mediated dUTP nick end-labelling (TUNEL) method (Enzo Kit, Life Sciences, NY, USA). After deparaffination and rehydration, tissue sections were digested with proteinase K (10 μg/ml) at 37° C. for 15 minutes. Following the application of an equilibration buffer, the sections were incubated in working strength TdT enzyme that contained deoxyuridine triphosphate (d-UTP)-biotin under a coverslip for 30 minutes at 37° C. After removal of the coverslip, the slides were incubated with streptavidin-conjugated alkaline phosphatase for 30 minutes at 37° C. and then with nitroblue tetrazolium for 30 minutes at 37° C. Sections of normal lymph nodes were used as positive control of the TUNEL method. In negative controls the TdT enzyme was omitted from the nucleotide mixture. The apoptotic signals, recorded as positive when either a diffuse-type or a granular-type nuclear dark blue staining was apparent, were also identified by the characteristic morphological appearance, the absence of significant nearby inflammation and the occurrence as isolated cells or clusters of apoptotic bodies.

Apoptotic hepatocytes were determined, blindly, by two different operators, checking apoptotic signals in twenty different fields (100× magnification). The data obtained were then expressed as percent number of apoptotic hepatocytes/1000 observed hepatocytes.

Cell proliferation: Bromo-Deoxyuridine (BrdU) Incorporation

The animals were injected i.p., 2 hours before killing, with 120 mg/kg b.w. of BrdU (Sigma, St Louis, Mo. USA). Liver samples, 5 mm thick, from the different lobes were fixed in buffered formalin, embedded in paraffin, sectioned at 4 μm, and stained with hematoxylin-eosin. BrdU incorporation was detected with a monoclonal anti-BrdU antibody (DAKO Corporation Carpinteria Calif. USA) at 1:20 dilutions. The reaction product was developed by 3-amino-9-ethylcarbazole Vectastain ABC kit PK 4002 (Vectors Laboratories Inc. Burlingame, Calif. USA). Hepatocyte proliferation was assessed in the lobular area of the entire liver Where the numbers of labelled and unlabelled nuclei were evaluated. A minimum of 1000 hepatocytes per liver lobe was counted by two different operators.

### Immunohistochemistry Evaluation of Stat-3-P

Blocks of paraffin-embedded liver samples from 70% PH rats treated with Alrp, rIGF-II or albumin and sacrificed 24 hours after surgery were sectioned at 4 μM on a standard rotary microtome (Reichert-Jung 1130/Biocut) and the sections were recovered from a water bath on APTS (3-amino-propyl-trimethoxy-silane) (Sigma Aldrich, St. Louis, USA) glass slides. The slides were deparaffinized in xylene for 1 hour at room temperature and rehydrated with decreasing concentrations of ethanol (10 min in ethanol 100%, 10 min in ethanol 95%, 10 min in ethanol 70%, 5 min in distilled water). In order to unmask antigenic sites, slides were exposed to microwaves at 700 W three times for 3 min in citrated buffer (pH=6.0). After three washes of 5 min in PBS (Phosphate Buffered Saline, pH=7.4), the slides where treated 10′ with Triton 0.25%. After three washes of 5 min in PBS, the endogenous peroxydase was then blocked in H₂O₂ in methanol (1:10 v/v) for 7 min at room temperature. After three washes of 5 min in PBS, the slides were placed in PBS containing 3% of Goat Serum (Sigma Aldrich, St. Louis, USA) (block solution) for 30 min at room temperature. The endogenous biotin was blocked with Avidin-Biotin Blocking Kit (Vector Laboratories, Burlingame, Calif.) and the sections were incubated in the block solution containing the primary polyclonal rabbit anti-P-Stat-3 antibody (Upstate, Lake Placid, N.Y.) diluted 1:300 at 4° C. o.n., as suggested by the manufacturer. Slides were then washed three times for 5 min in PBS and incubated for 1 hour at room temperature with PBS containing the secondary biotinylated anti-rabbit antibody (Vector Laboratories, Burlingame, Calif.) diluted 1:400, as suggested by the manufacturer. These are the best experimental conditions for an optimal visualization of P-Stat-3 in tissues (Chan K S et al, 2004). After three washes of 5 min in PBS, the slides were then incubated for 40 min at room temperature with a solution of peroxidase-linked ABC (ABC Kit, Vector Laboratories, Burlingame, Calif.), washed three times with PBS and then incubated for 14 min at room temperature with the chromogen 3-amino-9-ethyl carbazole (AEC Kit, Vector Laboratories, Burlingame, Calif.). After three washes of 5 min in PBS, sections were then mounted with Crystal/Mount (Biomeda Corp., Foster City, Calif.).

### Statistical Analysis

The results obtained were expressed as mean±Standard Deviation (M±S.D.). Statistical comparison among groups was determined using analysis of variance test (ANOVA). Where indicated, individual comparisons were performed using Student's test. Statistical significance was ascribed to the data when p<0.05.

1. The use of the Augmenter of Liver Regeneration (Alrp) as a regulator of apoptosis.

2. Use according to claim 1, for the preparation of a medicament for the regulation of apoptosis in a mammal.

3. Use according to claim 2, for the preparation of a medicament for the treatment and prevention of a disease wherein a regulation of apoptosis is beneficial.

4. Use according to claim 2 or 3, for the preparation of a medicament for the treatment and prevention of a disease provoked by troubles in the apoptosis control.

5. Use according to claims 2 to 4, for the preparation of a medicament for the treatment and/or prevention of a disease wherein an up-regulation of apoptosis is beneficial

6. Use according to any of claims 3 to 5, wherein said disease is selected from degenerative diseases.

7. A pharmaceutical or veterinary composition comprising Alrp as the active ingredient.

8. The use of Alrp as a marker for detecting the presence of a proliferative disease.

9. Use according to claim 8 for the preparation of a diagnostic agent.

10. The use of antibodies anti-Alrp for the preparation of a medicament for the treatment of a disease wherein a down-regulation of apoptosis is beneficial.

11. The use of antibodies anti-Alrp for the preparation of a diagnostic agent.

12. The use of Alrp for the preparation of cell lines.

13. The use of Alrp encoding sequence for the preparation of immortal cell lines.

14. The use of a transgenic non-human mammal transformed with an ALR gene sequence for experimental tests on apoptosis.

15. Use according to claim 14, as a model for experimental tests on apoptosis-linked diseases.
